(19) United States
(12) Patent Application Publication
(10) Pub. No.: US 2006/0058384 A1
(43) Pub. Date: Mar. 16, 2006

(54) Treatment of IGA nephropathy with omega-3 fatty acids

(75) Inventor: Ronald J. Hogg, Dallas, TX (US)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: PRONOVA BIOCARE, A.S., Lysaker (NO)

(21) Appl. No.: 11/221,827

(22) Filed: Sep. 9, 2005

Related U.S. Application Data
(60) Provisional application No. 60/608,540, filed on Sep. 10, 2004.

(51) Int. Cl. A61K 31/22; A61K 31/202

(52) U.S. Cl. 514/546; 514/560

(57) ABSTRACT

A statistically significant benefit is observed in the treatment of IgA nephropathy by administration of Omega-3 fatty acids in a dosing regimen dependent on the size of the patient.

### BACKGROUND OF THE INVENTION

1. Field of the Invention

Treatment of IgA nephropathy by administration of omega-3 polyunsaturated fatty acids according to a dosing regimen that is dependent on the weight and size of the patient being treated.

2. Description of Related Art

Omega 3 polyunsaturated fatty acids (“O3”), such as eicosapentaenoic acid (EPA) and docosahexaenoic acid (DHA), are beneficial in the treatment of various disorders including cardiovascular diseases, immune disorders, inflammation, renal disorders, allergies, diabetes and cancer. These fatty acids are also essential for the development of the brain and retina in humans. Eskimos in Greenland have lower serum cholesterol and triacylglycerol levels and lower incidence of cardiovascular disease owing to the relatively high intake of EPA from their diet. Studies with non-human primates and human newborns suggest that DHA is essential for the normal functioning of the retina and brain, particularly in premature infants. Other studies have shown that O3 can decrease the number and size of tumors and increase the time elapsed before the appearance of tumors.

Metabolically, EPA is an antagonist of the arachidonic acid cascade and competes with arachidonic acid as substrates for cyclooxygenase and lipoxygenase to produce eicosanoids. EPA is used for the synthesis of eicosanoids such as series-3 prostaglandins that ameliorate immunodysfunction. Arachidonic acid forms the series-2 prostaglandins that may impair the immune function. Diets containing high levels of n-6 fatty acids may increase the production of PGE2, decrease IL2 production, alter T cell response to IL2, inhibit macrophage collagenase synthesis, and enhance platelet aggregation. Feeding high levels of O3 will lead to substitution of some arachidonic acid by EPA and DHA. The PGE3 formed from EPA has less inflammatory effect than PGE2. IL 1 production is also lowered by O3 while IL 2 is increased. These changes in eicosanoid synthesis seen with O3 feeding are associated with an improved immunocompetence and a reduced inflammatory response to injury.

An O3 therapy for IgA nephropathy, using fish oil supplements, was first proposed 20 years ago. Hamazaki T, et al., “Eicosapentaenoic acid and IgA nephropathy”, Lancet 1984; 1:1017-8. However, resulting studies produced conflicting results leaving the role of this therapy unclear. See, e.g., Bennett W M, et al., “Treatment of IgA nephropathy with eicosapentanoic acid (EPA): a two-year prospective trial”, Clin Nephrol 1989; 31:128-31; Cheng I K P, et al., “The effect of fish-oil dietary supplement on the progression of mesangial IgA glomerulonephritis”, Nephrol Dial Transplant 1990; 5:241-6; Donadio J V, Jr, “Omega-3 polyunsaturated fatty acids: a potential new treatment of immune renal disease”, Mayo Clin Proc 1991; 66:1018-28; Pettersson E E, et al., “Treatment of IgA nephropathy with omega-3-polyunsaturated fatty acids: a prospective, double-blind, randomized study”, Clin Nephrol 1994; 41:183-90; Donadio J V, et al., “A controlled trial of fish oil in IgA nephropathy”, New Eng. J. Med., 1994; 331:1194-1198; Donadio J V, et al., Effects of n-3 Fatty Acids: Prevention and Treatment in Vascular Disease, Springer Verlag 1995; pps 173-180; Donadio J V, et al., “The long-term outcome of patients with IgA nephropathy treated with fish oil in a controlled trial”, J Am Soc Nephrol 1999; 8:1772-1777; Donadio J V, et al., “A randomized trial of high-dose compared with low-dose omega-3 fatty acids in severe IgA nephropathy” J Am Soc Nephrology 2001; 4:791-799.

The variability amongst the findings, and the resulting confusion has recently been illustrated in a meta-analysis. Dillon J J, “Fish Oil Therapy for IgA Nephropathy: Efficacy and Interstudy Variability” J Am Soc Nephrol 1997; 8:1739-1744. There is no accepted hypothesis explaining the inconsistent results.

### BRIEF SUMMARY OF THE INVENTION

An effective and reliable O3 therapy for IgA nephropathy involves adjusting the dose as a function of patient size. The use of O3 in a therapeutic formulation involves, among other things, compounding the formulation to facilitate the administration of at least a minimum effective dose, which is determined as a function of the size and/or body mass of the patient. An O3 therapy is shown to be effective and reliable when omega-3 fatty acids are administered in quantities of at least about 0.04 g per kg patient body mass.

For purposes of this disclosure, efficacy is determined on the basis of changes in the level of estimated glomerular filtration rate (“GFRₑₛₜ”) and/or level of proteinuria compared to baseline. The therapy can be supplemented by conjoint administration of prednisone.

Our investigation included alternate-day administration of prednisone or every day administration of omega-3 fatty acids in children and young adults with IgA nephropathy. Efficacy was evaluated by monitoring changes over time in GFR and the level of proteinuria. See, e.g., Donadio J V, et al., “Proteinuria patterns and their association with subsequent end-stage renal disease in IgA nephropathy”, Nephrol Dial Transplant 2002; 7:1197-2003 (showing GFR and proteinuria to be a good surrogate marker of progressive renal disease).

In a multicenter, placebo-controlled, double blind clinical trial, we evaluated the efficacy of a 24-month stable daily dose (4 grams per day) of a highly purified preparation of O3 (especially icosapent & doconexent). The primary analysis, which was based on a decrease of GFR to less than 60% of baseline did not show a statistically significant benefit from prednisone or O3.

We performed secondary analyses based on clinical response scores derived from changes in the level of proteinuria (defined by urine protein to creatinine ratios in first morning urine specimens) and GFRₑₛₜ. Composite Response Score, which represented changes in proteinuria and GFR scores were also calculated. The secondary analysis revealed that alternate day prednisone was effective. We also discovered that O3 therapy was effective, but only in patients who received a relatively high dose when expressed in terms of patient size and/or body mass.

The invention thus provides an IgA nephropathy treatment regimen wherein the O3 dose is calculated as a function of patient size and/or body mass to meet or exceed a minimum effective dose. Without wishing to be bound by any theory, it appears that O3 imparts a therapeutic effect only when concentrations within the body meet or exceed a certain minimum level. Thus, there is a threshold, or minimum effective dose, that must be administered in order to impart a therapeutic effect. Our studies show that the patient population as a whole will not respond to O3 therapy until the dose is adjusted to achieve administration of at least about 0.045 g O3 per kg body mass. Specifically, we found that administration of at least about 0.05 g/kg of Omacor® produced a substantial improvement in Composite Response Scores. Omacor® is a formulation of 90% omega-3 fatty acids of which 84% is a combination of the ethyl esters of EPA and DHA. Thus, the administration of 0.05 g/kg Omacor® is equivalent to the administration of 0.042 g EPA&DHA ethyl ester/kg body mass. One of skill in the art will appreciate that the advantages of the instant invention can also be realized by adjusting and monitoring the dose of O3 as a function of O3 concentration (and/or EPA/DHA concentration) within the blood.

The data further illustrate effective IgA nephropathy treatment regimens involving the administration of O3, and particularly EPA/DHA.

### BRIEF DESCRIPTION OF THE DRAWINGS

FIG. 1. Wide spectrum of phospholipid-fatty acid profiles in patients receiving icosapent/doconexent compared to placebo. Solid symbols represent: 1) omega-3/omega-6 (“O3/O6”)=total omega-3 content divided by omega-6 content in phospholipids; 2) EPA/AA=ratio of eicosapentaenoic acid/arachadonic acid; and 3) DHA/AA=ratio of docosahexaenoic acid/arachadonic acid, in patients receiving Omacor® (the O3 used in this study). The open symbols represent those ratios in patients receiving placebo capsules.

FIG. 2. Composite response scores observed at clinic visits at 6-month intervals in patients in the 3 treatment arms. The response scores were significantly better in the patients in the prednisone arm, but not in the O3 group as a whole.

FIG. 3. Composite response scores in the 3 treatment arms with responses in the patients who received icosapent/doconexent, being subdivided on the basis of plasma phospholipid EPA/AA ratios being above or below 0.38.

FIG. 4. Correlation between plasma phospholipid EPA/AA ratios and dosage of icosapent/doconexent expressed per kg/body weight.

FIG. 5. Composite response scores in the 3 treatment arms with the patients in the icosapent/doconexent arms being subdivided on the basis of their study drug dose being above or below 0.05 g/kg body weight.

### DETAILED DESCRIPTION OF THE INVENTION

The present invention provides a method, and a use of O3 in preparation of a medicament, for treating patients suffering from immune disorders, inflammation, renal disorders, allergies, diabetes, cancer, hypertriglyceridemia (“HTG”), and post-myocardial infarction by administering at least a minimum effective dose of O3 as calculated based on patient size and/or body mass. More particularly, the invention provides methods, and uses of O3 to produce medicaments, for the treatment of patients suffering from glomerular diseases, particularly IgA nephropathy.

In a preferred embodiment, the invention provides a method for treating a mammalian population suffering from, or at risk of, IgA nephropathy comprising administering to individuals within said population at least about 0.045 g/kg O3 to achieve a statistically significant improvement in GFR and/or proteinuria levels in the population. Preferred embodiments involve administration of at least about 0.042 g EPA&DHA ethyl ester per kg body mass; and preferably, about 0.042 g to about 0.13 g EPA&DHA ethyl ester per kg body mass. Another preferred embodiment involves administration of at least about 0.05 g EPA&DHA ethyl ester per kg body mass. Still another preferred embodiment involves the administration of about 0.05 g to about 0.11 g EPA&DHA per kg body mass. The GFR and proteinuria levels are measured as described elsewhere herein.

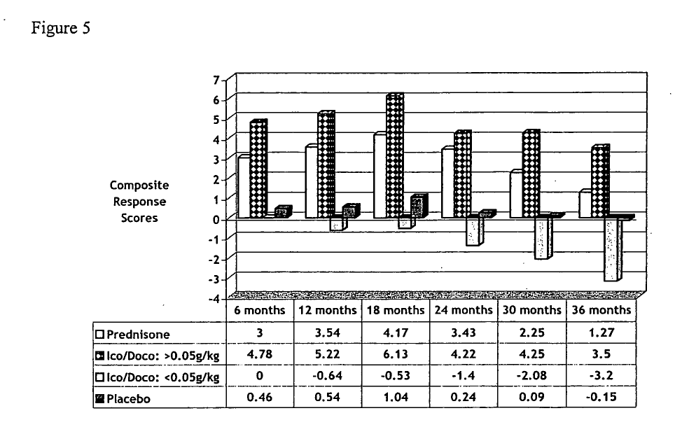

The method also achieves a significant improvement in Composite Response Scores in a patient population when measured against either placebo or an O3 dose of less than 0.04 g/kg body mass. In a preferred embodiment, the method achieves a Composite Response Score at least about 5-fold greater than placebo. The improved Composite Response Score is observed at least over the first 36 months of treatment. See, e.g., FIG. 5.

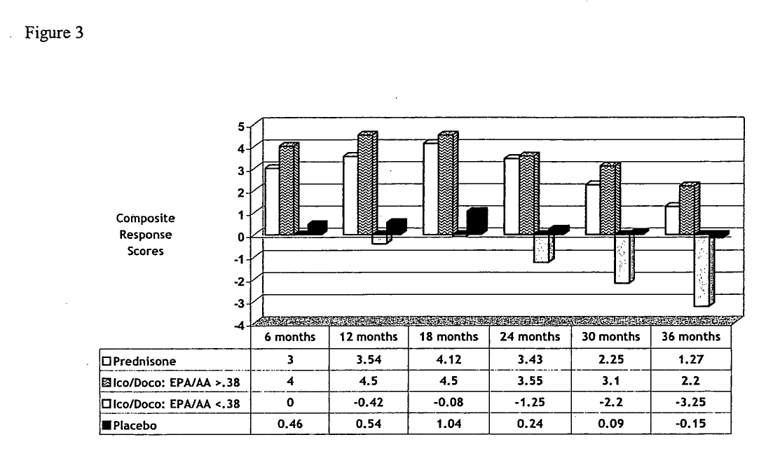

Likewise, a statistically significant improvement in Composite Response Scores is observed when the plasma phospholipid EPA/AA ratios of the patient population are elevated to at least about 0.38 by the administration of O3. Preferably, the method is employed such that the plasma phospholipid EPA/AA ratios are greater than 0.38. By adjusting the plasma phospholipid EPA/AA ratios to levels greater than 0.38, Composite Response Scores are at least twice those achieved with placebo, and can be about 4-fold or greater as compared with placebo. The same level of improvement, and even greater improvement, is achieved as compared with a patient population in which the plasma phospholipid EPA/AA ratios are less than 0.38. See, e.g., FIG. 3.

By patient (or mammalian) population is meant a diverse cross-section of individuals generally reflective of the size distribution of the population of patients suffering from, or at risk of, IgA nephropathy. The population should be sufficient in number as will achieve a normalized distribution of patient size generally consistent with the patient population. For purposes of this disclosure, and unless stated otherwise, a patient population is n≧6.

The invention further provides a method whereby O3 pharmaceutical formulations are administered to a patient population suffering from IgA nephropathy to achieve a Composite Response Score greater than about 1.5 during the first 36 months of treatment. Preferably, the method achieves a Composite Response Score greater than about 2.5. More preferably, the method achieves a Composite Response Score of about 3.5 or greater during the first 36 months of treatment. Still more preferably, the method achieves an average Composite Response Score of about 4.5 or greater during the first 36 months of treatment.

Another aspect of the invention involves the use of O3 in the manufacture of a medicament for administration to a patient population suffering from, or at risk of, IgA nephropathy to achieve a statistically significant reduction in the symptoms of IgA nephropathy within the population. The use described above further comprises the use of sufficient O3 to formulate a medicament to achieve administration of at least about 0.045 g O3/kg body mass. Preferably, the medicament comprises sufficient icosapent and doconexent to achieve administration of about 0.042 g to about 0.13 g EPA&DHA/kg body mass; and still more preferably, about 0.05 g to about 0.11 g EPA&DHA/kg body mass.

The O3 can be administered in a pharmaceutical formulation comprising a highly purified preparation of omega-3 fatty acids. One of skill in the art will recognize that the term omega-3 fatty acids includes natural sources such as fish oils, and those sources can include as many as 30 different omega-3 fatty acids. Preferably, the formulation comprises a substantial known quantity of icosapent and doconexent, i.e., the ethyl esters of eicosapentaenoic (EPA) and Docosahexaenoic acid (DHA).

A preferred pharmaceutical formulation of highly purified omega-3 fatty acids is Omacor®, commercially available from Pronova Biocare, Lysaker, Norway. Omacor® is about 90% omega-3 fatty acids, of which about 84% are the ethyl esters of eicosapentaenoic acid (EPA) and Docosahexaenoic acid (DHA).

The O3 pharmaceutical formulation can be administered by oral or parenteral routes, or as medicated applications (e.g., creams, lotions, suppositories, patches, etc.). Parenteral administration can be intravenous, subcutaneous, or intramuscular (including injections, implants, and transdermal devices). Further, the pharmaceutical formulations are compounded with conventional carriers, diluents, excipients, buffers, preservatives, biocides, and the like, and the selection of those additives will depend on various factors including the intended route of administration.

### EXAMPLE 1

This was a placebo-controlled, prospective clinical trial. After fulfilling all entry criteria, patients were assigned to one of three treatment arms, as described in our paper discussing the primary analysis of this study.

Randomization of patients was stratified by hypertensive status and blocked randomization was used within each stratum.

Estimation of GFR and level of proteinuria. The glomerular filtration rate (GFR) was estimated at 6 monthly intervals in each patient using standard age-appropriate formulas. Schwartz G J, et al., “A simple estimate of glomerular filtration rate in children derived from body length and plasma creatinine” Pediatrics 1976; 58:259-263; Schwartz G J, et al., “A simple estimate of glomerular filtration rate in adolescent boys”, J. Pediatr. 1985; 106:522-526; Cockcroft D W, et al., “Prediction of creatinine clearance from serum creatinine”, Nephron 1976; 16:31-41. Serum creatinine measurements were measured by HPLC. Welch M J, et al., “Determination of serum creatinine by isotope dilution mass spectrometry as a candidate definitive method”, Anal Chem 1986; 58:1681-1685; Rosano T G, et al., “Candidate reference method for determining creatinine in serum: method development and interlaboratory validation”, Clin Chem 1990; 36:1951-1955; and Quantimetrix Total Protein Determination (QuanT7test), Hawthorne, Calif., #40-02/87. The primary protein losses were estimated using the urine protein to creatinine ratios on first morning-voided specimen and a standard assay for protein measurements [Quantimetrix Total Protein Determination (QuanT7test), Hawthorne, Calif., #40-02/87] in a central laboratory.

Measurement of plasma phospholipids: Twenty-three of the patients who received icosapent/doconexent, and 13 of the patients who received placebo capsules had complete plasma phospholipid fatty acid profiles measured after 21-24 months of therapy. The methods that were used have been described in detail previously. Holub B J, et al., “Nutritional Regulation of Cellular Phosphatidylinositol”, Methods in Enzymology 1987; 141:234-245; Holman R T, et al., “Essential Fatty Acid Deficiency Profiles in Idiopathic Immunoglobulin A Nephropathy”, Am J Kidney Dis 1994; 23:648-654.

Adherence to the prescribed therapy regimens: Pill counts were conducted in the participating clinics to evaluate adherence to the prescribed dose of prednisone, icosapent/doconexent and placebo.

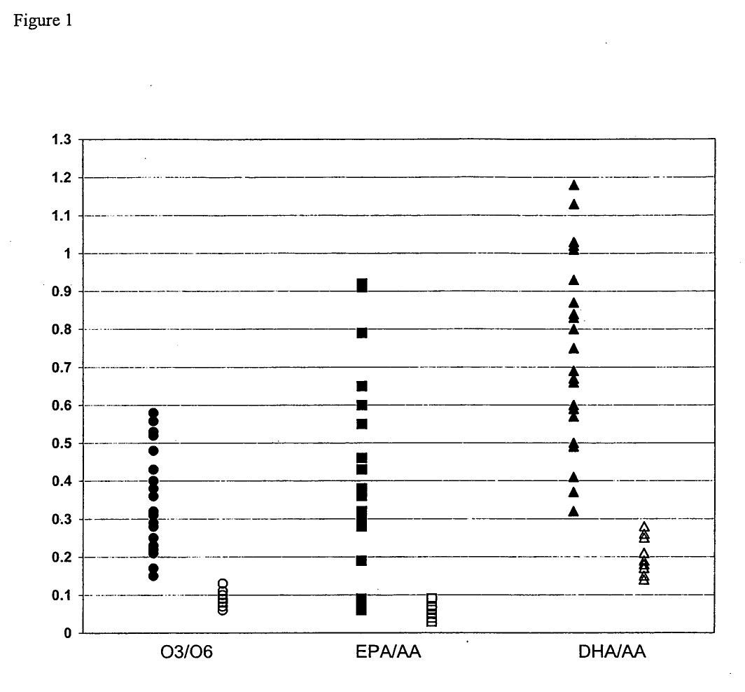

Statistical analysis: After it was noted that there was wide variation in the plasma phospholipid fatty acid profiles in patients receiving icosapent/doconexent (FIG. 1), a series of secondary analyses was conducted to determine the cause for this variability and to evaluate if the fatty acid profiles affected patient outcome. Clinical response scores were determined for each patient based on changes in the estimated GFR (−5 to +5) and urine protein/creatinine ratios (−5 to +5) at each 6 month visit up to 36 months. Composite response scores (−10 to +10) at each time interval were then determined based on the patients' combined GFR and UP/C Response Scores—with equal weight given to each (Table 1). Clinical outcome scores were then calculated based on the mean of the Composite Response Scores at 18 and 24 months. The dosage of the study drugs given to each patient was calculated as g/kg body weight and g/m² BSA.

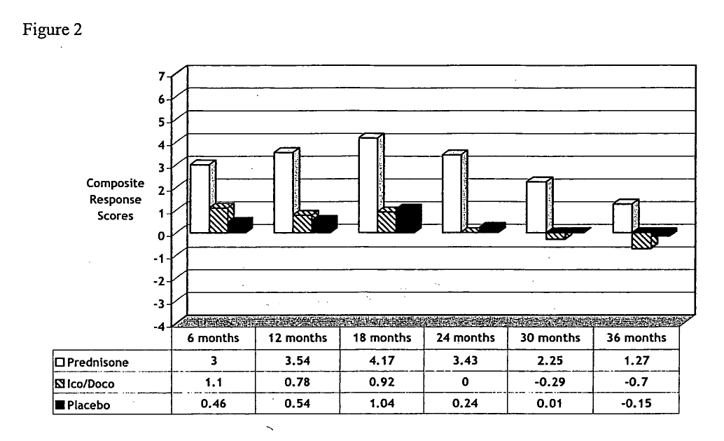

Clinical Response Scores: FIG. 2 shows the Composite Response Scores in the 3 treatment groups that were observed every 6 months between 0 and 36 months. There was a better response to prednisone than the other approaches, especially up to 24 months. No apparent response was noted in patients in the icosapent/doconexent arm. The Composite Response Scores were then re-evaluated with the patients treated with icosapent/doconexent being subdivided into 2 sub-groups on the basis of their phospholipid fatty acid profiles. FIG. 2 shows the statistically significant relationship that was detected between the clinical response scores and the phospholipid EPA/AA ratios above and below 0.38. Similar results (not shown) were obtained when these patients were sub-divided on the basis of O3/O6 ratios above and below 0.31.

Relationship between clinical response/outcome scores, phospholipid fatty acids and dosage of icosapent/doconexent: As shown in FIG. 1, the phospholipid omega-3/omega-6 ratios ranged from 0.17 to 0.58 (mean=0.34) and the EPA/AA ratios ranged from 0.06 to 0.91 (mean=0.42) in the 23 patients in the icosapent/doconexent arm who had phospholipid fatty acids measured, versus 0.06-0.13 and 0.03-0.09 in the placebo patients. There was no relationship between the omega-3/omega-6 and EPA/AA ratios and the adherence to therapy (r=0.09, r=0.20.)

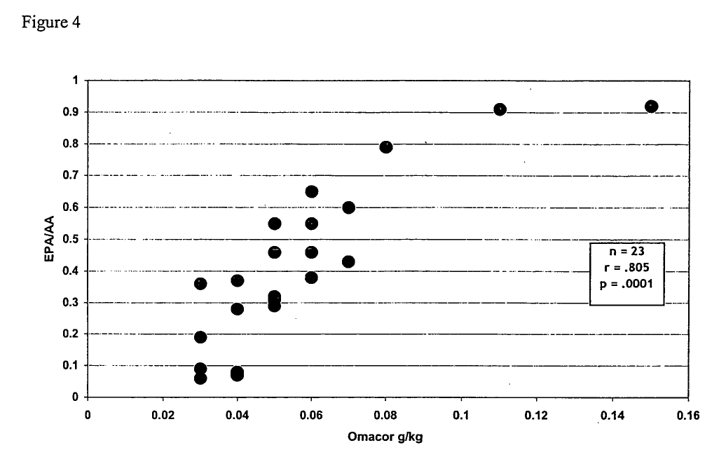

The phospholipid omega-3/omega-6 and EPA/AA ratios were closely correlated with icosapent/doconexent dosing as g/kg (r=0.68 and 0.80, p=0.0001) and g/m² (r=0.69 and 0.82, p=0.0001). The relationship between the EPA/AA levels and dosing per kg body weight is shown in FIG. 4. The phospholipid EPA/AA and DHA/AA ratios were also closely correlated (r=0.85, p=0.0001). The Composite Response Scores correlated with omega-3/omega-6 (r=0.53, p=0.009, EPA/AA (r=0.59, p=0.003), dose in g/kg (r=0.55, p=0.006) and dose/m² BSA (r=0.54m, p=0.008), but not adherence to therapy (r=0.21). The scores that were observed in patients receiving more than or less than 0.05 g/kg icosapent/doconexent are shown in FIG. 5. A summary of the correlations is provided in Table 2.

Secondary analyses of the results obtained in this trial provide convincing evidence that a two-year course of each of the study drugs was associated with an improved outcome, especially when this was defined in terms of proteinuria and the dose of the medications was calculated on the basis of patient size.

Recent large clinical trials of patients with various glomerular diseases associated with proteinuria have concluded that changes in the level of proteinuria provide the best surrogate marker for response to therapy. However, the lack of uniformity in the entry levels of proteinuria in the patients in the three treatment arms in our trial compromised our ability to use raw proteinuria data in this analysis. This led to the decision to develop clinical outcome scores, based on both GFR and UP/C ratios, separately and combined, to provide a better mechanism for studying the interaction between the study drugs and the disease activity in individual patients. The composite outcome scores that we have used places equal weight on changes in GFR and UP/C ratios. This was chosen arbitrarily in the absence of evidence to do otherwise in our relatively short-term study.

Description of Terms Used to Describe Clinical Responses/Outcomes

UP/C Clinical Response Score—Changes in urine protein/creatinine ratio:
±1=±20% change in UP/C ratio from baseline
±2=±21-40% change in UP/C ratio from baseline
±3=±41-60% change in UP/C ratio from baseline
±4=±61-80% change in UP/C ratio from baseline
±5=±≧81% change in UP/C ratio from baseline

Positive numbers indicate improvement (i.e., decrease) in UP/C

GFRₑₛₜ Clinical Response Score—Changes in GFRₑₛₜ:
±1=±10% change from baseline
±2=±11-20% change from baseline
±3=±21-30% change from baseline
±4=±31-40% change from baseline
±5=±≧41% change from baseline

Positive numbers indicate improvement (i.e., increase) in GFR

Additional Measures To Evaluate Outcomes:

Composite Score—Combination of GFRₑₛₜ score and UP/C score:
Best=+10
Worst=−10

Clinical Outcome Score

Mean of composite scores at 18 and 24 months of treatment

TABLE 2
Correlation between clinical outcome scores, phospholipid fatty acid profiles,
dosage of treatment factored for body size, and adherence to therapy

| | | | | | Treatment |
|---|---|---|---|---|---|
| | PL-EPA/AA | PL-O3/O6 | Treatment | Treatment | Dose % of |
| | Ratio | Ratio | Dose (g/k) | Dose (g/m²) | Adh-Rx |
| UP/C Outcome | r = .643 | r = .595 | r = .538 | r = .549 | r = .154 |
| Score | p = .001 | p = .003 | p = .008 | p = .007 | p = .483 |
| GFR Outcome | r = .480 | r = .409 | r = .582 | r = .528 | r = .092 |
| Score | p = .020 | p = .053 | p = .003 | p = .008 | p = .668 |
| Composite | r = .589 | r = .532 | r = .554 | r = .538 | r = 0.21 |
| Outcome Score | p = .003 | p = .009 | p = .006 | p = .008 | p = 0.33 |
| PL-EPA/AA | — | — | r = .805 | r = .818 | r = 0.20 |
| Ratio | | | p = .0001 | p = .0001 | p = 0.37 |
| PL-O3/O6 | — | — | r = .683 | r = .694 | r = 0.09 |
| Ratio | | | p = .0001 | p = .0001 | p = 0.70 |

Abbreviations:
UP/C=urine protein to creatinine ratio
GFR=glomerular filtration rate (note that positive score is indicative of improvement in all clinical outcome scores)
Treatment=4 grams per day
EPA=eicosapentaenoic acid
AA=arachadonic acid

What is claimed is:

1. A method for treating a mammal suffering from, or at risk of, IgA nephropathy comprising administering to said mammal at least about 0.04 g/kg omega 3 polyunsaturated fatty acids, and repeating said administration to achieve a statistically significant improvement in GFR and/or proteinuria levels in the population.

2. The method of claim 1, wherein prednisone is conjointly administered with the omega 3 polyunsaturated fatty acids.

3. The method of claim 1, wherein the mammal is a human.

4. The method of claim 1, wherein the administration of omega 3 polyunsaturated fatty acids is repeated daily.

5. The method of claim 4, wherein the daily adminstration is repeated over the course of at least about two years.

6. A method for treating a mammal suffering from, or at risk of, IgA nephropathy comprising administering to said mammal at least about 0.045 g/kg omega 3 polyunsaturated fatty acids, wherein said omega 3 polyunsaturated fatty acids comprise at least about 84% by weight of a combination of ethyl esters of eicosopentaenoic acid and docosahexaenoic acid, and repeating said administration daily.

7. The method of claim 6, wherein the mammal is a human.

8. The method of claim 6, wherein prednisone is co-administered to the mammal every other day.

9. A method for treating a mammal suffering from, or at risk of, IgA nephropathy comprising administering to said mammal at least about 0.042 g of a combination of ethyl esters of eicosopentaenoic acid and docosahexaenoic acid per kilogram body mass.

10. The method of claim 9, wherein the mammal is a human.

11. The method of claim 9, wherein prednisone is co-administered to the mammal every other day.

12. The method of claim 9, wherein the combination of the ethyl esters of eicosopentaenoic acid and docosahexaenoic acid are administered at a dose of about 0.042 to about 0.13 g per kilogram body mass.

13. The method of claim 9, wherein the combination of ethyl esters of eicosopentaenoic acid and docosahexaenoic acid are administered at a dose of about 0.05 to about 0.11 g per kilogram body mass.

14. The method of claim 9, wherein the administration of ethyl esters of eicosopentaenoic acid and docosahexaenoic acid is repeated daily for about two years or more.

15. A method for treating a mammal suffering from, or at risk of, IgA nephropathy comprising administering to said mammal omega 3 polyunsaturated fatty acids to achieve plasma phopholipid EPA/AA ratios of at least about 0.38.

16. The method of claim 15, wherein the mammal is a human.

17. The method of claim 15, wherein the omega 3 polyunsaturated fatty acids are administered to said mammal at a dose of at least about 0.042 g of a combination of eicosopentaenoic acid ethyl ester and docosahexaenoic acid ethyl ester per kilogram body mass.

18. The method of claim 15, wherein the omega 3 polyunsaturated fatty acids are administered to said mammal daily at least until the plasma phopholipid EPA/AA ratio in said mammal exceeds 0.38.

19. A method for treating a patient suffering from inflammation, a renal disorder, or hypertriglyceridemia by administering to said patient at least about 0.045 g/kg omega 3 polyunsaturated fatty acids, wherein said omega 3 polyunsaturated fatty acids comprise at least about 84% by weight of a combination of ethyl esters of eicosopentaenoic acid and docosahexaenoic acid, and repeating said administration daily.

20. The method of claim 19, wherein the administration of omega 3 polyunsaturated fatty acids is repeated daily.

21. The method of claim 20, wherein the administration is repeated daily for a period of about two years or more.
